(19) United States
(12) Patent Application Publication
(10) Pub. No.: US 2019/0255298 A1
(43) Pub. Date: Aug. 22, 2019

(54) SLOTTED GUIDEWIRE PROTECTOR AND ADVANCING DEVICE AND METHOD

(71) Applicant: Redsmith, Inc., Walnut Creek, CA (US)

(72) Inventors: James D. Mitchell, Walnut Creek, CA (US); Andrew A. Thoreson, Long Lake, MN (US)

(73) Assignee: Redsmith, Inc., Walnut Creek, CA (US)

(21) Appl. No.: 16/402,373

(22) Filed: May 3, 2019

Related U.S. Application Data
(63) Continuation of application No. PCT/US17/60063, filed on Nov. 4, 2017.
(60) Provisional application No. 62/419,426, filed on Nov. 8, 2016.

(51) Int. Cl. A61M 25/09

(52) U.S. Cl. CPC A61M 25/09041; A61M 2025/09116; A61M 2025/09183

(57) ABSTRACT

A guidewire advancing device used in connection with medical devices for providing intravascular access for advancing substantial length of guidewire used to place such medical devices within the anatomical structure. A method of managing substantial length of guidewire for use with medical devices. The guidewire advancing device includes a longitudinally extending wire guide which is removably secured to the medical device and which includes a longitudinally extending channel. The guidewire extends the length of the channel to be advanced along the channel.

### CROSS-REFERENCE TO RELATED APPLICATIONS

This application is a continuation of PCT/US17/60063, filed Nov. 4, 2017 which claims priority to U.S. Pat. App. No. 62/419,426 filed Nov. 8, 2016, both entitled Slotted Guidewire Protector and Advancing Device and Method, the disclosures of which are incorporated herein by reference.

### FIELD OF THE INVENTION

The present invention is directed, generally, to a guidewire protector and advancing device used in connection with medical devices for providing intravascular access into a blood vessel, including veins, arteries, or other anatomical structure for advancing the guidewire used to place such medical devices within the anatomical structure. More specifically, the guidewire protector and advancing device rapidly and easily advances substantial lengths of wire for rapid and effective intravascular placement of the medical device, such as through the proximal end of an access needle integrated with a catheter.

### BACKGROUND OF THE INVENTION

Intravascular (IV) access is a mainstay of medical care, and provides a conduit for delivery of life-saving medications, fluids, and nutrition. Furthermore, venous access allows the aspiration and sampling of blood for diagnostic purposes to learn more about the medical patient. IV access may be within a peripheral vein, a central vein, or an intermediate placement such as a peripherally inserted central catheter (PICC) or midline/extended dwell peripheral IV (EDPIV). Intra-arterial access is also commonplace, and the archetype of arterial access is that of the well-described arterial “art” line. Rather than serving as a conduit for the administration of fluids as is seen in venous lines, arterial lines are most frequently employed for diagnostic purposes in the invasive, yet accurate, collection of vital signs and blood gases. Finally, arterial access also enables percutaneous introduction of vascular devices, namely hemostatic sheaths, for the subsequent introduction and exchange of various catheters, wires, and other endovascular devices.

Barriers to effective and safe intravascular access include limitations in patient anatomy, patient medical condition, potential loss of sterility, and user proficiency. Anatomical barriers include size and depth of the vessel, tortuosity or lack of a sufficient inline segment, vessel spasm, and lack of good direct or ultrasound visualization. When patients require intravascular access, they are often in an unhealthy state, which may further complicate the creation of durable access, including smaller vessels in the setting of dehydration or systemic illness, as well as concern for potential bleeding complications in the ill and anticoagulated patient. Sterility may be broken dependent on the preparation and positioning of the patient's access site, but may often be related to operator error while try to control an unwieldy object, or make multiple device exchanges. All the aforementioned barriers may be further complicated by individual skill and training. Therefore, desirable attributes of a vascular access device include an intuitive device whose components, namely the wire, are user-friendly and sufficiently long enough to obtain the necessary amount of purchase inside a vessel to maximize trackability, and ultimately, the technical success, sterility, and safety of introducing an integrated catheter.

### SUMMARY OF THE INVENTION

It is, therefore, advantageous to provide a guidewire protector and advancing device for rapidly, efficiently, and effectively advancing substantial lengths of guidewire through a needle for medical device placement within an anatomical structure, such as an artery or vein, while also rendering the procedure as minimally invasive as possible. Although originally designed for use with a vascular access catheter, the device could also be used to facilitate access into other body cavities such as the thorax for insertion of chest tubes or the abdomen for insertion of paracentesis or nephrostomy catheters or drains. The guidewire protector and advancing device may be used on conjunction with any medical device employing a guidewire, including U.S. Ser. No. 15/008,628 for Rapid Insertion Integrated Catheter and Method of Using an Integrated Catheter, which is incorporated herein by reference.

The present invention overcomes shortcomings of the prior art by providing a novel slotted guidewire protector and advancing device which facilitates guidewire insertion into a target vessel for gaining vascular access, such as performing an endovascular procedure. For demonstration purposes, the guidewire advancing device according to the present invention is used with a tubular catheter body configured to receive a hollow needle for receiving the guidewire. The guidewire advancing device includes a slotted wire guide, a guidewire stay, a needle hub and a clamp. More specifically, the wire guide is a generally tubular member having a longitudinally extending channel with an open upper edge configured for receipt of a guidewire. The channel configuration permits the guidewire to be advanced distally, along the length of the channel wherein the proximal portion of the guidewire may be advanced outside of the channel. The guidewire stay is positioned on the proximal end of the guidewire and is manipulated by the treating physician for advancing the guidewire. The brace connects the wire guide to the catheter and is easily removed therefrom. The distal portion of the guidewire may be engaged with the needle during use, or it may be pre-assembled with the needle wherein advancement of the guidewire advances the wire distally from the needle tip for intravascular placement.

The method of using the device permits the treating physician to effectively, accurately, and rapidly advance the guidewire for intravascular placement of a medical device. The length of the guidewire is managed as it is advanced along the length of wire guide channel. Upon placement of the needle within the target vessel, the physician may advance the guidewire by advancing the guidewire stay which passes adjacent to the open side of the wire guide channel. The guidewire stay advances until engaging the needle hub, or in the fully advanced position at the distal conical portion of the wire guide. The guidewire stay is designed to prevent loss of the wire through the access needle and into the patient's vasculature, however, a separate embodiment could include a simple guidewire without a stopper. These and other objectives are met by the present invention.

### BRIEF DESCRIPTION OF THE DRAWINGS

FIG. 1A is a top view of the guidewire protector and advancing device;

FIG. 1B is a perspective view of the guidewire protector and advancing device according to the present invention engaged with a catheter and having the guidewire fully received therein;

FIG. 2 is a side elevational view of the guidewire protector and advancing device of FIG. 1 along with integrated catheter;

FIG. 3 is a top plan view of the guidewire protector and advancing device of FIG. 1 along with integrated catheter;

FIGS. 4A-B are an enlarged top view, and enlarged perspective view of the brace for attaching the guidewire guide to the catheter and integrated catheter;

FIGS. 5A-D are a bottom plan, and side elevational view of the guidewire protector and clamp, respectively; and

FIG. 6 is a perspective view of the guidewire protector and advancing device in an operative position;

FIG. 7 is a perspective view of the guidewire protector and advancing device in a fully engaged position for treatment;

FIG. 8 is a perspective view of the guidewire protector and advancing device in an operational position for treatment;

FIG. 9 is a perspective view of a guidewire stay on the proximal end of the guidewire according to another aspect of the invention for use with the advancing device.

### DETAILED DESCRIPTION OF THE INVENTION

The present invention will now be described in detail hereinafter by reference to the accompanying drawings. The invention is not intended to be limited to the embodiments described; rather, this detailed description is provided to enable any person skilled in the art to make and practice the invention.

As used herein, the terms “proximal” and “distal” (excluding horizontal cross-sections) are used to refer to the axial ends of the assembly. The term “proximal end” refers to the end closely adjacent the user of the assembly and the term “distal end” refers to the end of the assembly to be intravascularly positioned.

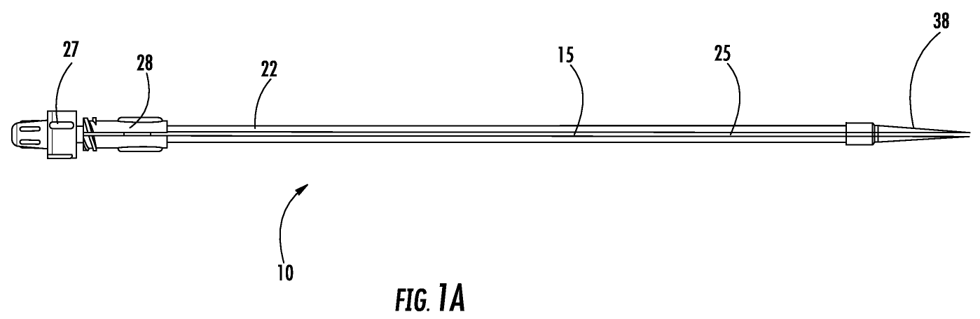

The guidewire advancing device 10, as shown in FIG. 1A is used in connection with a catheter 12, as shown in FIG. 1B, for containing, advancing, and guiding the guidewire 15 distally to attain intravascular access for catheter 12 placement. A coupling 21 selectively connects the guidewire advancing device 10 and catheter 12. By way of an exemplary use of the guidewire advancing device 10, the venous catheter 12 as illustrated is a triple lumen catheter. It is within the scope of the present invention, however, to provide a single lumen and one or more additional non-needle lumens. A central lumen extends from the proximal end to the distal end of the catheter 12 body. Two additional lumens are provided and each lumen is independent and not communicative with the other lumens. The catheter 12 further includes, in the illustrated embodiment, entry ports 17, 18, and 19, which communicate with a respective lumen for the administration of or extraction of fluids or blood. As is known in the art, entry ports 17, 18, 19, are selectively manipulated to occlude the lumens. Extension tubes connect the entry ports to a respective lumen and may be of any desired length. Side clamps, as shown, may also be provided along the extension tubes as is known in the art to control the fluid flow through the lumen. As to one aspect of the present invention, a coupling 21 joins the extension tubes to the proximal end of the catheter 12 to establish communication between the extension tubes and the respective lumen. The coupling 21 is provided with a pair of radially extending wings which are advantageously used to further secure the catheter in place with sutures or adhesive-type devices applied to the skin (such as StatLock®). A brace 20 cooperates with the coupling 21 to removably secure the guidewire protector and advancing device 10 to the catheter 12 as explained more fully below.

For the purposes of its use with the rapid insertion venous catheter, the wire 15 is preassembled into the proximal end of the venous access needle 13. Alternatively, the guidewire may be inserted into the access needle after initiation of the procedure, that is, it is not “pre-loaded” into the needle. The device 10 may also be used in a manner as to be contained within the housing of a peripheral IV (PIV) or midline/extended dwell peripheral IV (EDPIV) device, or as a mechanism to be used in conjunction with any type and configuration of a peripheral or central line. One skilled in the art of vascular access and minimally invasive procedures could also envision a device that could contain this mechanism not only for peripheral or central venous access, but also for arterial access as is seen in arterial lines for invasive vital sign monitoring, hemostatic vascular sheaths for venous/arterial procedures, or access components, such as peel-away sheaths, for example. Additionally, the guidewire device 10 described herein could be used in conjunction or incorporated in a device used for percutaneous access into any cavity as is seen in image-guided drain/catheter placements into an abscess, the renal collecting system, biliary system, or any space or potential space of the human body.

The catheter 12 is a disposable, single use device that is made of a biocompatible material. The needle and guidewire may be made of known materials, such as steel, nitinol, or a composition including one or both. The guidewire 15, according to one aspect, is formed of a flexible material to accommodate anatomical complications such as complex and tortuous vasculature. Commonly used materials are a polymeric coated or metallic wire.

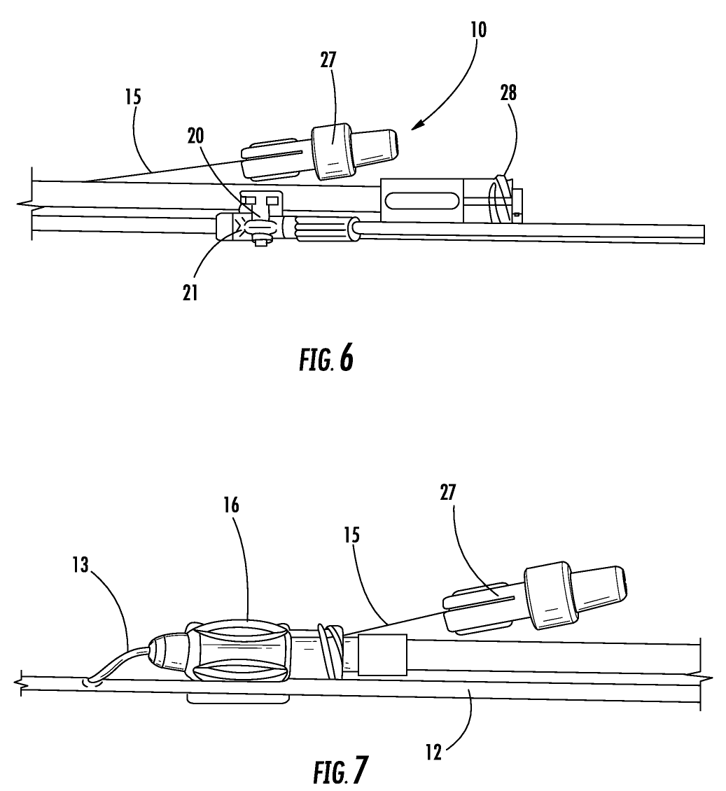

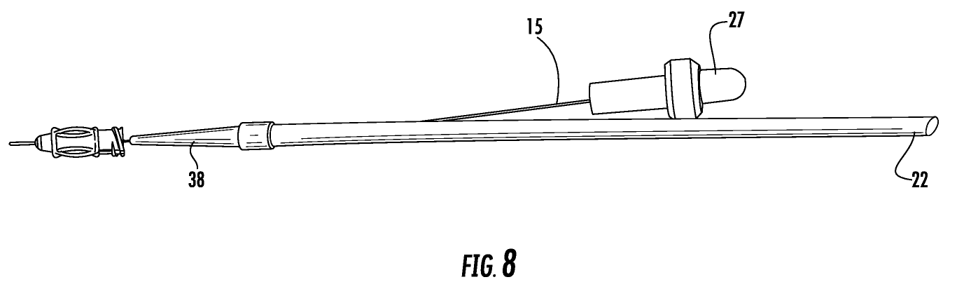

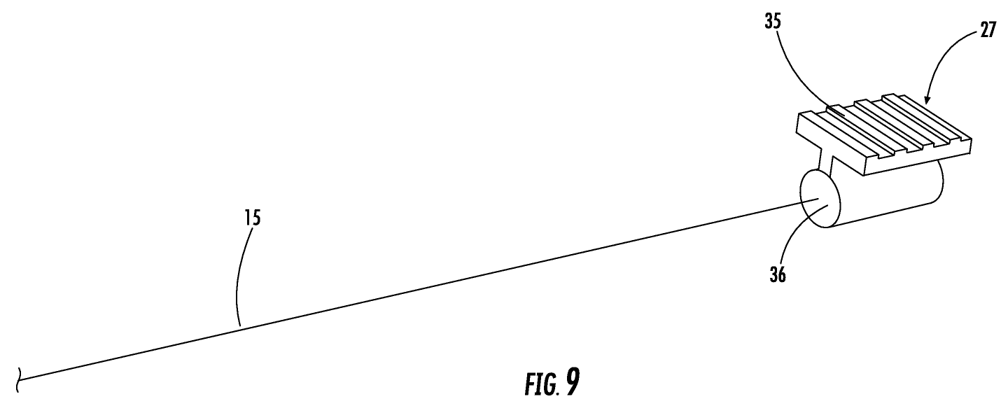

The needle 13 has an insertion tip and is hollow and may be formed of a surgical grade stainless steel, such as an 18-22 gauge, as is common in the art. FIG. 7 illustrates the needle 13 in a fully engaged position that is, fully extended into the vascular structure for treatment. As shown in FIG. 7, the needle 13 is inserted into the needle side port 29 and extends along the length of the catheter lumen associated with the side port 29. The needle 13 extends through a distal aperture on the distal tip of the catheter 12. The distal aperture is preferably sized in accordance with the needle gauge wherein only minimal tolerances are provided for smooth trackability. As shown in the various Figures, the guidewire advancing device 10 is used with a catheter side port 29. The present invention, however, is not limited to a side port and may be employed with conventional catheters, particularly when substantial length guidewire is required, wherein the needle is inserted into the central lumen.

As shown, the guidewire 15 extends along the length of the hollow needle 13. The proximal end of the guidewire 15 preferably includes visible marks at regular intervals, for example, 1 cm, to indicate the depth of the wire. This avoids placement too deep within the vasculature, but of sufficient depth for catheter placement. The guidewire 15 may be formed of material known in the art, such as a composite with a nitinol core to provide a blend of stiffness and flexibility, for effective torque control and venous navigation.

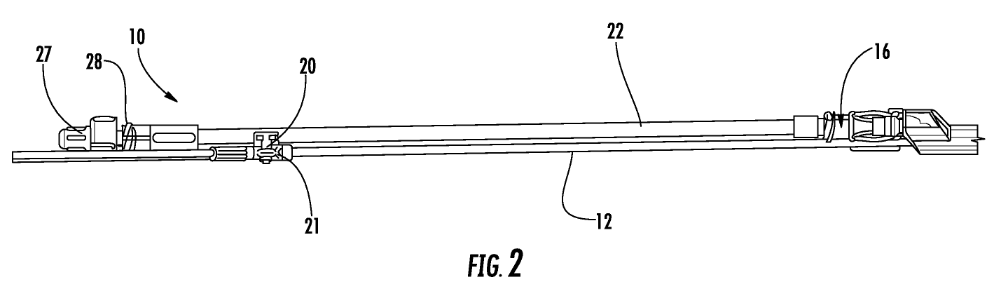

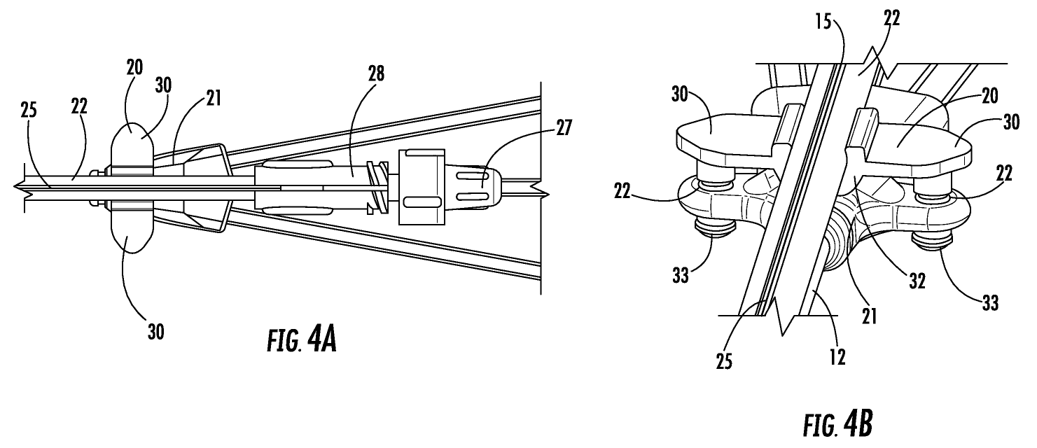

The guidewire advancing device 10, as shown in FIGS. 1A, 1B, 5A-5D, includes a wire guide 22 in the form of a generally cylindrical body. Along its outer surface, the wire guide 22 defines a longitudinally extending channel 25 best shown in FIGS. 1 and 3. The channel 25, preferably, extends substantially the entire length of the wire guide 22 body. It is within the scope of the present invention, however, that the channel 25 not extend entirely to its proximal or distal end. The channel 25 has a depth sufficient to receive varying gauges of guidewire 15.

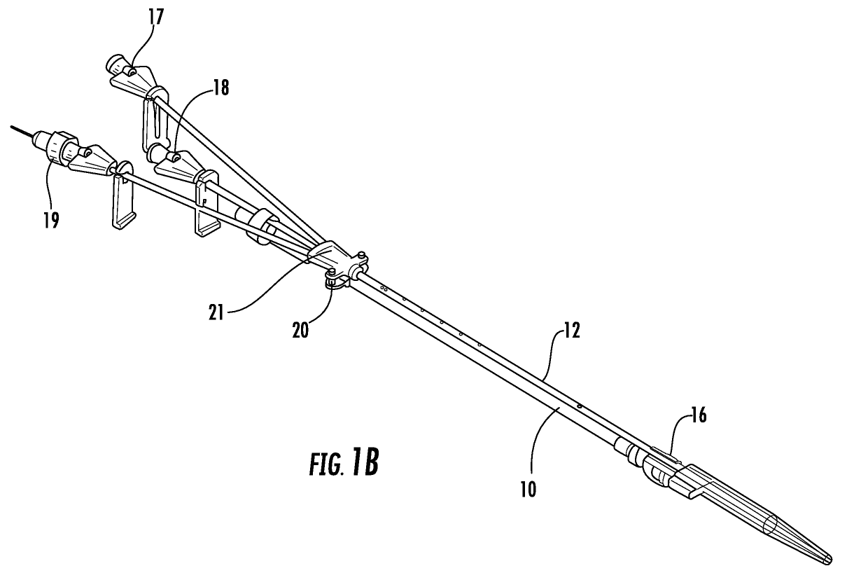

The guidewire advancing device 10 also includes a tapered distal end 38, a proximal cap 28, and a guidewire stay 27 positioned on the proximal end of the guidewire. The proximal cap 28 is also slotted and defines a channel to allow passage of the guidewire 15. The proximal cap 28 and the guidewire stay 27 communicate with each other and are held in place by either static friction or a screwing thread mechanism analogous to a Luer Lock device. The tapered distal end 38 is configured for receipt by the needle hub 16. The distal end 38 may be integrally formed with the wire guide 22 or, as shown, separately formed wherein the wire guide 22 is inserted therein. The needle hub 16, shown in FIGS. 2-3 defines an aperture on its proximal end configured to receive the wire guide 22 and includes a distal aperture through which the needle 13 extends during use. The needle 13 thereby extends through the needle hub 16. The guidewire stay 27 defines a longitudinally extending aperture for receiving the proximal end of the guidewire 15 and is configured to cooperate with the proximal cap 28 to prevent loss of the wire through the needle 13 when inserted for treatment.

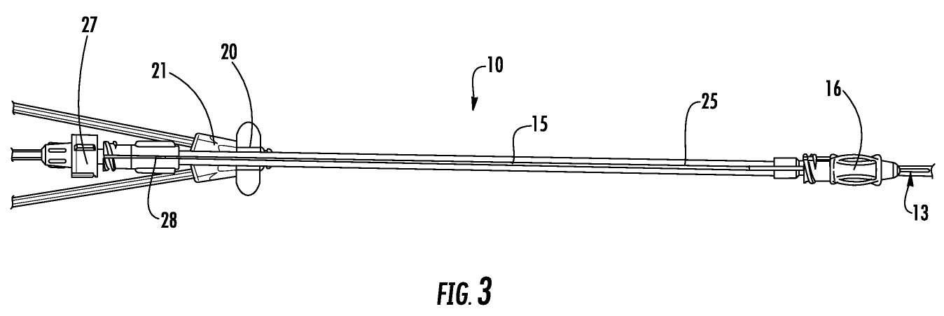

The guidewire advancing device 10 further includes a brace 20. Brace 20 shown in FIGS. 4A, 4B, 5A-5D includes a pair of lateral wings 30 for easy manipulation of the brace 20 which extend outwardly from a central body. The central body 32 defines an arcuate, concave seat configured to receive the wire guide 22. The central body seat provides a frictional connection with the wire guide 22 wherein sufficient tolerances of the central body aperture and the wire guide permit the wire guide 22 to be “snapped” or pressure fit into the seat 32 and similarly removed therefrom. A pair of posts 33 extend from the wings 30 to cooperate with the corresponding apertures 22 of the catheter coupling 21 to secure the wire guide 22 to the catheter 12. The wire guide 22 is thereby secured due to appropriate tolerances between the coupling apertures 22 and posts 33 and brace 20 wherein sufficient forces may be used to readily remove the wire guide 22 with easy manipulation. Preferably, wire guide 22 may be pulled from the brace 20 once needle and guidewire insertion is complete. The guidewire advancing device is shown integrated with a central venous catheter for demonstration purposes, but it is within the scope of the invention to have the guidewire advancing device 10 integrated with any vascular access or other invasive diagnostic or interventional medical device. As such, the brace 20 may take on a configuration other than the design shown.

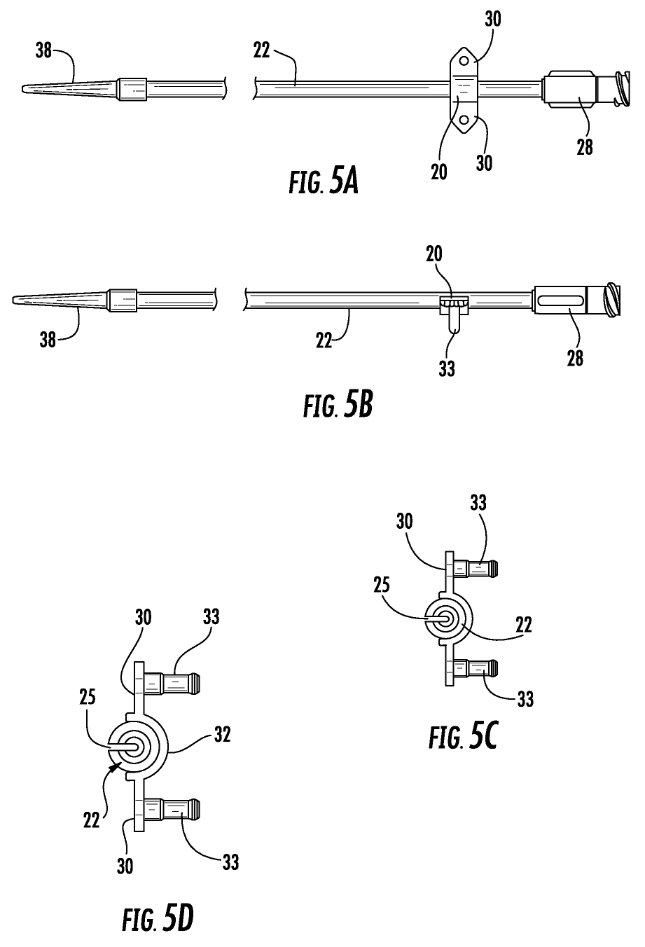

The wire guide 22 may be of any desired length, such as substantially the length of the catheter 12 as shown. The channel 25 is open on its proximal end wherein the guidewire 15 extends along the length of the channel 25 and the guidewire stay extends proximally from the wire guide 22 as shown in FIGS. 1 and 3. As the guidewire 15 is advanced distally, the guidewire curves to permit the guidewire stay 27 to pass above the open channel as handled by the physician advancing the guidewire 15. This is shown in FIG. 6. As the guidewire is fully advanced, the guidewire stay 27 engages the proximal cap 28 preventing loss of the guidewire within the patient.

As shown, the wire guide 22 is linear. It is within the scope of the present invention, however, to provide the wire guide in a non-linear configuration, such as curvilinear, or a combination of portions which are linear and non-linear.

In operation, the guidewire 15 is preferably preloaded in the wire guide 22. The wire guide tip 38 articulates with the needle hub 16. The wire guide 22 is then connected to the catheter 12. The brace 20 of guidewire advancing device 10 mates with the catheter coupling 21, such as by a snap fit. The needle 13 is then inserted into the target vessel for treatment.

For treatment, the guidewire 15 is advanced distally to extend beyond the distal tip of the needle 13 and further into the target vessel for treatment. The length of the guidewire 15 is restrained by but slideable along the wire guide channel 25. Once positioned, the physician, utilizing the guidewire stay 27 as a handle, distally advances the guidewire 15. As it is advanced, the guidewire 15 curves upwardly, out of the channel 25, as shown in FIG. 6 wherein the proximal portion of the guidewire 15 extends above the wire guide 22. In the fully advanced or engaged position, the guidewire stay 27 contacts the needle hub 16, thereby preventing loss of the guidewire 15 within the patient.

FIG. 19 illustrates a guidewire stay 27 connected with the guidewire 15 proximal end according to another aspect of the presently disclosed invention. The guidewire stay 27 includes a tab 35 configured for manipulation with the user's finger and a guidewire proximal stop 36 secured to the proximal end of the guidewire 15 for supporting the tab 35. The stop 36 is configured for receipt within the wire guide 22 and for movement along the longitudinal channel 25 of the advancing device 10. The stop 36 prevents loss of the wire through the needle 13 when inserted for treatment.

Upon completion of guidewire 15 placement within the target vessel as necessary for the medical procedure, the wire guide 22 may be removed by applying appropriate force to disconnect the connection between the catheter coupling 21 and the brace 20. The catheter 12 may then be advanced along the length of the guidewire 15 until placed within the target vessel. The guidewire 15 and the needle 13 may then be withdrawn, leaving the catheter 12 in-dwelling within the vessel.

While exemplary embodiments have been shown and described above for the purpose of disclosure, modifications to the disclosed embodiments may occur to those skilled in the art. The disclosure, therefore, is not limited to the above precise embodiments and that changes may be made without departing from its spirit and scope.

What is claimed is:

1. A guidewire advancing device for use with a medical device and for advancing a guidewire having a proximal and distal end to achieve intravascular placement of the medical device comprising:
a wire guide longitudinally extending between a proximal and distal end;
a channel defined by an outer surface of said wire guide and extending a predetermined distance between said proximal and distal ends of said wire guide, said channel being configured for receipt of the guidewire and for movement of said guidewire along the length of said channel;
a proximal cap positioned along the proximal end of the wire guide and defining a seat for receipt of the guidewire;
a guidewire stay connected to the proximal end of the guidewire; and
a brace for connecting said wire guide to the medical device.

2. A guidewire advancing device according to claim 1 wherein said guidewire stay is configured to selectively cooperate with said proximal cap.

3. A guidewire advancing device according to claim 1 wherein said guidewire stay defines an aperture for receipt of the proximal end of said guidewire.

4. A guidewire advancing device according to claim 1 wherein said brace includes a seat for receipt of said guidewire advancing device.

5. A guidewire advancing device according to claim 4 further comprising a coupling wherein said coupling cooperates with said brace for securing said advancing device to the medical device.

6. A guidewire advancing device according to claim 5 further comprising at least one post extending between said brace and said coupling for clamping said guidewire advancing device to said medical device.

7. A guidewire advancing device according to claim 5 wherein said brace is removable from said coupling.

8. A guidewire advancing device according to claim 1 wherein said guidewire stay includes a finger tab and a stop wherein said stop is configured for receipt within said channel and connected to the proximal end of said guidewire.

9. A guidewire advancing device according to claim 1 wherein said channel predetermined length extends substantially the length of said advancing device from said proximal to said distal ends thereof.

10. A guidewire advancing device according to claim 1 wherein said advancing device length defined between said proximal and distal ends is substantially the length of the medical device selectively connected by said coupling.

11. A method of using a guidewire advancing device for advancing a guidewire to achieve intravascular placement of a medical device comprising the steps of:
positioning the guidewire advancing device adjacent an outer surface of the medical device;
connecting the guidewire advancing device to said medical device with a brace;
advancing the guidewire along the length of the guidewire advancing device within a channel defined by said guidewire advancing device so as to advance the guidewire into the medical device and through a needle for obtaining vascular access for medical device placement; and
removing the brace and the guidewire advancing device when vascular access is achieved.

12. A method according to claim 11 further comprising the step of providing a guidewire stay on a proximal end of the guidewire and a proximal cap on a proximal end of said guidewire advancing device and wherein said guidewire stay and said proximal cap cooperate to prevent loss of the guidewire proximal end.
